(19) United States
(12) Patent Application Publication
(10) Pub. No.: US 2025/0018096 A1
(43) Pub. Date: Jan. 16, 2025

(54) BREAST PUMP SYSTEMS AND METHODS

(71) Applicant: Willow Innovations, Inc., Mountain View, CA (US)

(72) Inventors: Jenny Teng, Belmont, CA (US); Jason Chamberlain, Campbell, CA (US); Becky Scanlon, Los Altos Hills, CA (US); Elizabeth Ho, Milpitas, CA (US); Marvin Patel, Aptos, CA (US); John Chang, Los Altos, CA (US)

(21) Appl. No.: 18/895,708

(22) Filed: Sep. 25, 2024

Related U.S. Application Data
(63) Continuation of application No. PCT/US23/16482, filed on Mar. 28, 2023.
(60) Provisional application No. 63/324,204, filed on Mar. 28, 2022.

(51) Int. Cl. A61M 1/06

(52) U.S. Cl. CPC A61M 1/064

(57) ABSTRACT

Systems and methods for pumping milk from a breast, wherein the milk is expressed from the breast under suction and milk is expulsed from the pumping mechanism to a collection container. Various sealing points are provided between components of the assembly for sealing purposes and to accomplish overall pumping targets.

### FIELD OF THE DISCLOSURE

The present disclosure generally relates to portable breast pump systems and methods for collecting milk from a breast of a nursing mother.

### BACKGROUND OF THE DISCLOSURE

As more women become aware that breastfeeding is the best source of nutrition for a baby, and also offers health benefits to the nursing mother, the need is increasing for breast pump solutions that are user-friendly, quiet, discrete and versatile for use by a nursing mother in various situations. This is particularly true for the working mother, who is away from the home for eight to ten hours or more and needs to pump breast milk in order to have it available for her baby, but it is also a requirement for many other situations where the mother is away from the privacy of the home for an extended period, such as during shopping, going out to dinner or other activities.

Although a variety of breast pumps are available, a number are awkward and cumbersome, requiring many parts and assemblies and being difficult to transport. Hand pump varieties that are manually driven are onerous to use and can be inconvenient to use. Some powered breast pumps require an AC power source to plug into during use. Some systems are battery driven, but draw down the battery power fairly rapidly as the motorized pump continuously operates to maintain suction during the milk extraction process. Many of the breast pumps available are clearly visible to an observer when the mother is using it, and many also expose the breast of the mother during use.

There is a continuing need for a small, portable, self-powered, energy efficient, wearable breast pump system that is easy to use, that mimics natural nursing, and is discrete by not exposing the breast of the user and nearly unnoticeable when worn.

It can alternatively or additionally be desirable to provide a breast pump system including a volume equalization and diffusing chamber, the volume equalization and diffusing chamber defined by the lid and flange and a milk collection container assembly configured to receive pumped milk.

There is thus a continuing need for a breast pump system that is effective and convenient to use that embodies such features. The present disclosure addresses these and other needs.

### SUMMARY OF THE DISCLOSURE

Briefly and in general terms, the present disclosure is directed toward breast pump systems or methods. The system includes breast contacting structure and a reusable collection or storage container or assembly, and pumping structure that delivers milk from a breast to the collection assembly. The method involves pumping milk from a breast and delivering the pumped milk into the collection assembly or storage container.

According to one aspect of the present disclosure, a system for pumping milk from a breast includes one or more of: a skin contact member or flange configured to form a seal with the breast; a conduit or tube in fluid communication with and connected to the skin contact member; a driving mechanism configured to establish a vacuum profile within the conduit; an external shell; a reusable milk collection container; and a non-transitory computer readable medium having stored thereon instructions executable by a computing device to cause the computing devices to perform functions associated with and directed by the instructions; wherein the external shell comprises a compartment; wherein the skin contact member, the conduit and the driving mechanism are received in the compartment of the external shell; wherein the milk collection container and the shell are shaped and configured to be contoured to the breast of a user.

In various of the disclosed embodiments, the system defines a breast profile. The natural breast profile is contemplated to fit comfortably and conveniently into a bra of a user and to present a natural look. As such, the profile is characterized by having a non-circular base. Moreover, like natural breasts, the profile of the device or system is contemplated to define one or more asymmetric curves and off-center inertial centers. The breast engaging portion or flange embodies structure that curves and wraps back and around the pump in a direction away from the breast to thereby present comfortable, soft and round surfaces to the breast. In one aspect, the system defines a breast enhancement system for enlarging the appearance of the user's breast.

In at least one embodiment, the system functions by operating a control system that tracks internal pressure of the system against a known waveform. In this regard, the waveform can be a vacuum waveform indicative of pressures applied to a breast, and can define a sine wave fluctuating between about 60 mmHg of vacuum to a vacuum from about 120 mmHg to about 250 mmHg, or other desirable or useful waveform.

In one or more embodiments, the system includes a controller that accomplishes real time pressure control inside the system.

In one or more embodiments, the system includes a controller providing automated compliance sensing and response.

In one or more embodiments, the system includes a non-contact pressure sensing arrangement that does not touch the skin or the milk inside the tube while accurately determining internal pressure of the tube.

In one or more embodiments, the system includes one or more controllers that automatically detects one or more of letdown, overfill and flow.

In one or more embodiments, the system can be adapted to visualize a user's data and trends as it relates to volume (from each breast and total), and number of sessions on several dimensions (per day, per week and per month). Data and analytics can also be provided on pumping session.

In at least one embodiment, the flange or skin contact member, the conduit, the driving mechanism, the external shell and the milk collection container are all contained within a cup of a brassiere.

In at least one embodiment, the system is battery powered, the system comprising a battery, wherein the battery is received in the compartment of the external shell.

In at least one embodiment, the milk collection container comprises a one-way valve that permits milk inflow into the milk collection container but prevents milk backflow from the milk collection container to the conduit. In one embodiment, the collection container or container assembly includes an extra part, valve or fitment that is attached thereto and facilitates creating a seal with the container to establish a closed system. Moreover, in one or more embodiments, the container is reusable and can be removed from the breast pump system.

In at least one embodiment, the system further includes a milk collection container, wherein the milk collection container is in fluid communication with the conduit. In one or more aspects, the container is reusable and can include a reusable or hinged lid as well as a removable or otherwise incorporated liner.

In at least one embodiment, the breast pumps system includes a pressure sensor that senses the pressure at the breast. The sensor is a non-contact sensor in that it is not in direct contact with one or more of the breast or the fluids pumped by the breast pump system.

In at least one embodiment, the skin contact member includes: a breast contact portion configured and dimensioned to fit over and form a seal with a portion of the breast; and a nipple receiving portion extending from the breast contact portion.

According to another aspect of the present disclosure, a method of operating a system for pumping milk includes one or more of: providing the system comprising a skin contact member configured to form a seal with the breast, a conduit in fluid communication with and connected to the skin contact member; a driving mechanism, and a controller configured to control operation of the driving mechanism; sealing the skin contact member to the breast; operating the driving mechanism to generate predetermined pressure cycles within the conduit; and monitoring by the controller; measuring or calculating pressure within the conduit.

In at least one embodiment, the predetermined pressure cycles comprise extraction pressure cycles, and the controller modifies operation relative to an amount of milk entering the conduit, to maintain predetermined pressures during the extraction pressure cycles.

In at least one embodiment, the predetermined pressure cycles comprise latch cycles, wherein upon determination that milk has entered the conduit or after a predetermined period of time, the controller operates the roller compression members to achieve predetermined extraction pressure cycles, wherein the predetermined extraction cycles differ from the predetermined latch cycles by at least one of maximum suction level, cycle frequency or waveform shape. Latch can be created within a nipple receiving portion of a flange or at the engagement between the breast and the flange proximal of the nipple receiving portion. Moreover, in one or more embodiments, the system includes structure or functions to recognize when a user is done pumping, or includes structure or functions such that when there is a loss of vacuum recognition which allows the user to easily end a pumping session by simply pausing and pulling the device off of the breast.

In one or more embodiments, the system provides a variance in vacuum waveforms as needed, selected and/or directed by a system controller. In one aspect, such variance in waveforms accomplish controlling latch vacuums such that a user directly or through an app or the system automatically alternates between latching and not latching, or a level or degree of latch can be varied during a pumping session. Latch can be varied from for example 60 mmHg for a number of cycles and then that latch can vary and that latch can be reduced to zero or slightly positive or otherwise mimic suckling patterns of a child, be responsive levels of mobility, or comfort needs generally or due to cracked or sore nipples or other health needs. One objective can be to provide the user with a more natural feeling the same facilitating a breast to reach let down and/or express more milk over a pumping session.

In one particular embodiment, the breast pump system includes one or more of a flange configured and dimensioned to form a seal with the breast, the flange including a nipple tunnel, a lid sized and shaped to receive the flange, a diaphragm, a first chamber, the first chamber defined by the lid and diaphragm, a pump assembly configured to flex the diaphragm and create a vacuum within the first chamber, a volume equalization and diffusing chamber, the volume equalization and diffusing chamber defined by the lid and flange and a milk collection container assembly configured to receive pumped milk, wherein the diaphragm functions to create a vacuum within the first chamber and the volume equalization and diffusing chamber conditions the vacuum being applied within the nipple tunnel. In one aspect, the diaphragm is frusto-conical in shape. The diaphragm is configured to cooperate with the lid, the flange and a pump assembly and to provide multiple areas of sealing of the diaphragm to one or more of the lid, the flange and pump assembly. As the breast pump system operates, such points of sealing are enhanced as the engagement between the diaphragm and the other structures is increased through vacuum pressures generated by the pump assembly.

These and other features of the disclosure will become apparent to those persons skilled in the art upon reading the details of the systems and methods as more fully described below.

### BRIEF DESCRIPTION OF THE DRAWINGS

FIG. 1A is a perspective view, depicting an alternative embodiment of a breast pump system.

FIGS. 1B-C are exploded views, depicting the breast pump system of FIG. 1A.

FIG. 1D is a cross-sectional view, depicting components of the breast pump system of FIG. 1A.

FIG. 1E is a cross-sectional view, depicting features of the breast pump system of FIG. 1A.

FIG. 1F is a bottom view, depicting features of a container for use with the breast pump system of FIG. 1A.

FIG. 1G is a side view, depicting the container of FIG. 1F as part of an assembly.

### DETAILED DESCRIPTION OF THE DISCLOSURE

Before the present systems and methods are described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are now described. All publications or applications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a sensor” includes a plurality of such sensors and reference to “the pump” includes reference to one or more pumps and equivalents thereof known to those skilled in the art, and so forth.

The publications or applications discussed herein are provided solely for their disclosure prior to the filing date of the present application. The dates of publication provided may be different from the actual publication dates which may need to be independently confirmed.

Various details of related systems can be found in U.S. application Ser. Nos. 15/083,571 (now U.S. Pat. No. 9,539,376), 15/361,974; 15/362,920; and Ser. No. 15/406,923 (now U.S. Pat. No. 10,434,228) each filed Jul. 21, 2015; Ser. No. 16/050,201 filed Jul. 31, 2018; Ser. No. 17/095,892 filed Nov. 12, 2020; and PCT/US21/62241 filed Dec. 7, 2021, each of which are hereby incorporated herein, in their entireties, by reference thereto.

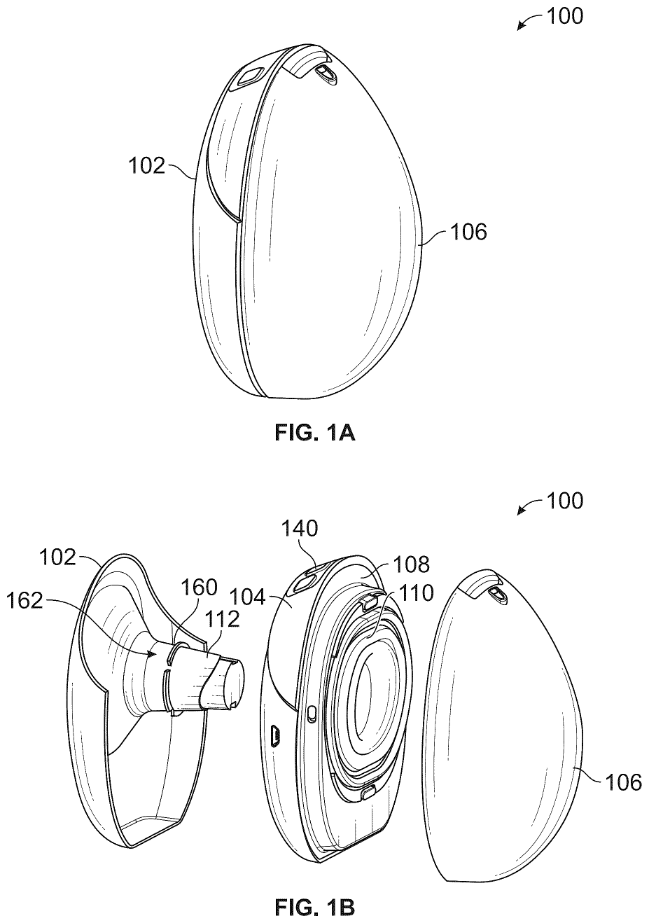

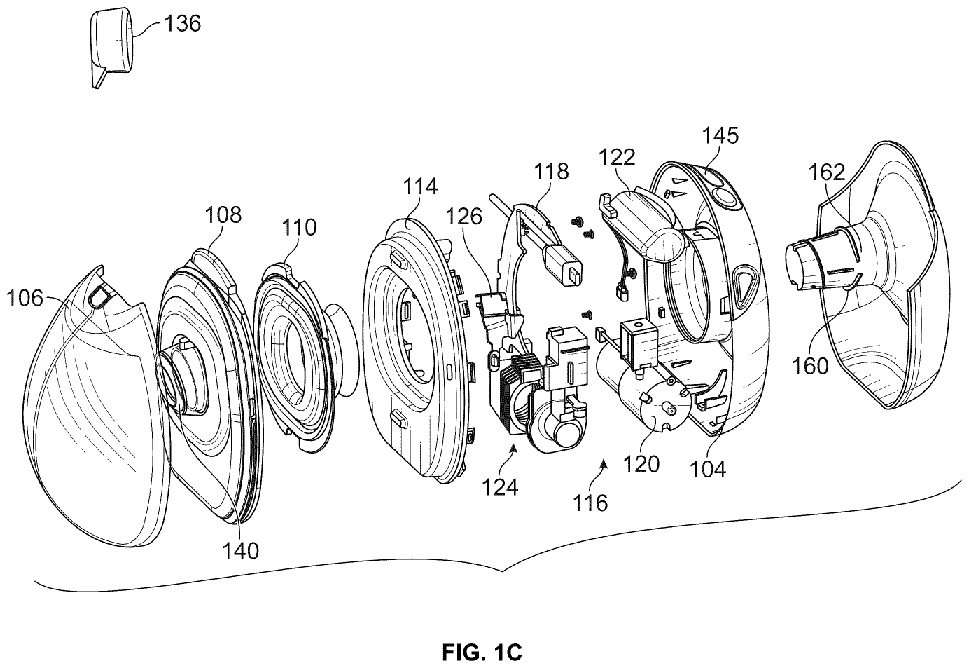

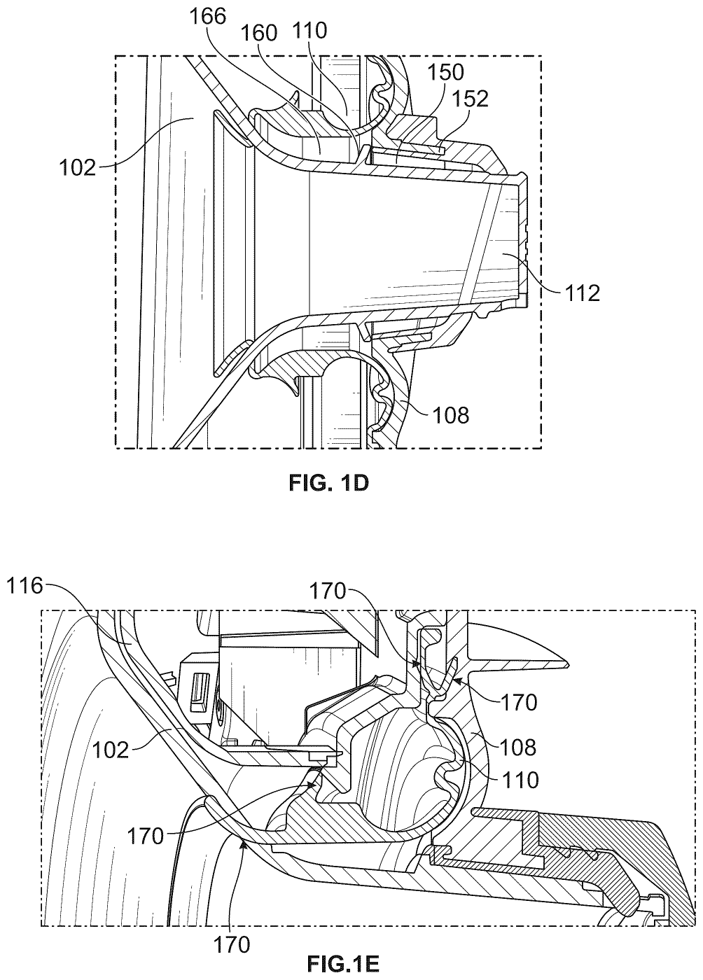

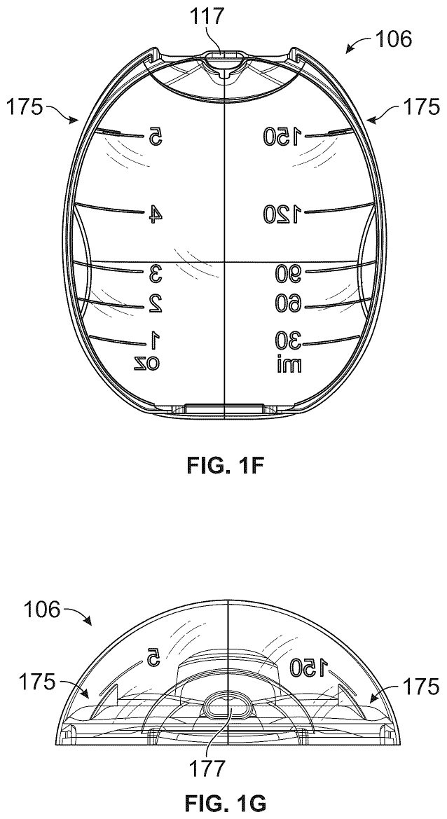

In one embodiment, there is shown an approach to a breast pump system 100 in FIGS. 1A-D. With reference to FIG. 1B, the breast pump system includes a flange 102 sized and shaped to receive a breast of a user, the flange 102 configured to engage a back housing 104, and the system further includes a milk container 106 that engages and connects to a lid 108. The back housing 104 and the lid 108 are configured to connect to each other and sized and shaped to retain further internal components of the breast pump system 100. Attached and extending through the lid 108 is a diaphragm 110 that includes a central opening. When breast pump system 100 is in its assembled form (FIG. 1A), a nipple tunnel 112 of the flange 102 extends laterally through the back housing 104, diaphragm 110 and the lid 108. Moreover, when assembled, a majority of a volume of the breast pump system 110 is below the nipple tunnel 112 of the flange 102 so that the majority of the volume can be retained discreetly within a bra or clothing of a user. In one approach, the diaphragm 110 can assume and defines an overall frusto-conical profile. Moreover, the system includes gentle curved surfaces and the milk container 106 defines a natural breast shape.

As best seen in the fully exploded view of FIG. 1C, configured and sandwiched between the back housing 104 and the lid 108 is a front housing 114 to which components of a pump assembly 116 are attached. The pump subassembly 116 includes a PCB 118, a motor assembly 120, a solenoid and a battery assembly 122. Adapted to be configured about or adjacent the motor assembly 120 is a muffler assembly 124 that includes a sleeve and muffler components that cooperate to reduce the sound generated by the operation of the pump and motor assemblies. A latch assembly 126 is further provided to assist in holding the pump subassembly 116 components in place between the back housing 104 and the lid 108.

Additionally, configured between the lid 108 and the container 106 can be a duckbill valve 136 that mates with a center hole 140 formed in the lid 108. In operation, the pump subassembly 116 functions to cause the diaphragm 110 to flex or pulsate according to a controlled pattern to thereby create desired waveforms of vacuum within the nipple tunnel 112. This action causes letdown and the flow of milk from a user's breast. The milk is transported from the nipple tunnel 112 through the duckbill valve 136 and into the milk container 106. A control panel 145 is configured on a top outside surface of the breast pump system 100 and via its communication with the pump subassembly 116 is configured to provide the user with means for controlling the operation of the pump 116. One or more of the pump features and functionality described below or herein, including a controller providing direct or remote control and data collection, can be incorporated into the breast pump system 100 so that the needs and desired pump operation is provided to the user. Moreover, the system 100 can be configured to function to evacuate air from the container 106 before, during or after use, and the motor assembly 116 can be programmed or controlled to achieve target vacuum levels such as pulling more vacuum after levels dips to a certain degree.

Notably, this approach to the breast pump system 100 includes a volume equalization and diffusing chamber 150. The volume equalization and diffusing chamber 150 one or more conditions the vacuum created by the pump so that the vacuum being applied to a breast is equally distributed within the nipple tunnel 112 or functions as a sound dampener. As best seen in FIG. 1D, the volume equalization and diffusing chamber 150 is formed by an outer surface of the nipple tunnel 112 and an inner surface of a generally tubular portion 152 extending from the lid 108. An annular angled wall 160 is formed about the outer surface of the nipple tunnel 112, the annular wall 160 including a plurality of cut-outs 162 in selected locations to provide an opening to the volume equalization and diffusing chamber 150 (See also FIG. 1C). In one embodiment, there are three such cut-outs 162 formed in the annular wall 160, but fewer or more cut-outs of varying shapes can also be utilized. As such, the pulsating or flexing action of the diaphragm 110 caused by its interaction with the pump subassembly 116 creates a desired amount and character of vacuum in a first chamber 166. The volume equalization and diffusing chamber 150 then conditions this vacuum prior to the vacuum entering the nipple tunnel 112 to create a more controlled target vacuum. Thus, the vacuum generated by the pump subassembly 116 is first applied within the first chamber 166, and this vacuum is conditioned by the volume equalization and diffusing chamber 150 so that the conditioned and controlled vacuum can be applied to breast tissue as desired and directed.

With reference to FIG. 1E, there are shown multiple sealing points 170 created through the engagement of the diaphragm 110 with one or more of the lid 108, the flange 102 and the pump assembly 116. As shown, there are four sealing points 170 between such structure but fewer or more sealing points are contemplated. Additionally, it is to be understood that as the breast pump system 110 operates, one or more of the sealing points 170 are enhanced through the deflection or elastic deformation of the diaphragm 110 at the areas that the diaphragm engages one or more of the lid 108, pump assembly 116 or flange 102. Accordingly, during operation, the diaphragm self-seals at engagement points or areas.

Turning now to FIGS. 1F-G, there is shown one specific embodiment of a milk collection container 106. To facilitate inhibiting the flow of collected milk along a perimeter of the container 106, the container is equipped with splash ribs 175. The splash ribs 175 can extend along a length of a perimeter of the container to thus direct milk away from a spout 177 form in the container. In this way, as the user of the breast pump system moves or otherwise the breast pump is moved about, pumped milk is directed away from the spout to thus avoid leakage.

In one or more additional aspects that can be incorporated into the above disclosed approach, as milk enters the system, the suction level decreases (pressure increases). Feedback provided by pressure monitoring via pressure sensor provides input to a feedback loop that maintain the desired vacuum (pressure) within the system by compensating for the changes in pressure that occur to changing amounts of milk in the system. In one particular embodiment, a pressure sensor provides information relative to latch of the breast pump to a breast, and such information can be communicated via BLUETOOTH or the like to control pump function to achieve a desirable or target latch. In one aspect, the user can control the degree of latch of the breast pump to the breast and/or turn latch off or on.

Moreover, in one or more embodiments, system firmware can be configured to alter pump noise based upon suction strength such as more noise is generated by the pump system when there is an increase in suction strength and less noise when there is a decrease in suction strength. This is useful for a breast pump system that does not have different level of sounds associated with pumping suction levels but there is an expectation of a user that different sounds would be generated for different pumping suction levels. Additionally, system firmware can provide for varying perceived patterns and/or speeds of the pump as a user adjusts from one level of suction to another, again to match expectations of a user where a pump system would not otherwise generate such perceived patterns or speeds. In yet another aspect, firmware can provide a rolling waveform of suction to thus provide a more quiet pumping action.

In general, real-time pressure control of the breast pump system can be managed by a controller (not shown) of one embodiment of the system. The controller tracks pressure and moves the pump motor either to influence the pressure in the direction of its choosing. If there is vacuum in the system that vacuum can be increased as the volume of the tube is manipulated. The pump controller applies these principles, sensing the current pressure and then adjusting to generate a pressure target. By doing this repeatedly in real time, the system can create a controlled vacuum waveform that matches waveforms desired to be applied to a user's nipple. In one particular aspect, the system generates a varying vacuum on the breast, the varying vacuum being a repeating waveform from low vacuum to a higher vacuum then returning to the low vacuum. Here, the waveform period is divided into sections of specified duration such that there can be one section with a duration of the waveform period or if there are multiple sections, the sum of each section duration equals the waveform period and the vacuum for each section is specified by a mathematic function. Such an approach allows for control of the rate of vacuum change when increasing and decreasing vacuum.

The system can further be provided with automated letdown detection. The pump can sense when it is full of fluid and responds accordingly by switching between pumping and letdown when fluid has begun to flow. In one approach an algorithm incorporated into the system can operate to look at the ratio of maximum and minimum of a target wave in the pump and compare that against the output of the pump. The result is a unit-less but very reliable sensing of system compliance. This can be tuned to trigger an internal event when the compliance crosses some known values that represent when the system is full of fluid. Any other measurement of compliance can be used in an equivalent way.

In another approach to letdown detection, it is noted that pushing air does not generate the same forces as pushing fluid. Tracking the force generated during a purge can also give a strong indication of when the system is full of fluid. An event can be generated to track this such that when the force of a purge crosses some known threshold the system can be said to be full of fluid rather than air. This approach may involve less tracking of data and less tuning that is subject to change with pump design or breast tissue. In yet another approach, letdown detection can be based upon tracking flow. That is, when flow begins, letdown must have occurred and when a small volume of flow has been collected the system can switch to pumping. Further, letdown can be tracked by looking at the relative rate of change of vacuum measured to motor position. Note that this relative rate of change is a measurement of compliance. As this ratio goes up in magnitude, it can be concluded that the system is filling with fluid.

A system controller or system software and/or firmware controls the action of the pump in real time, responsive to pre-determined latching and production targets or schemes as detected by the pressure sensor. The firmware can be written so that such targets can be approached at various speeds, sometimes relatively quickly and other times more slowly or gently to thereby provide multiple stimulation and expression levels. Thus, for example, latch can be achieved taking alternatively more gradual or quicker approaches, and there can be controls determining the level at which latch is achieved. Various levels of suction can be present during expression as well.

Upon commencing milk extraction, the pump assembly can function in the same manner as in latching, but in a manner that follows an extraction waveform determined by the selected extraction pumping determined in real time by system controls which are responsive to the pressure sensing assembly. Continued cyclical operation of the pump assembly increases the pressure in the system to establish a positive pressure to drive the contents (milk) through a one-way valve to the milk container assembly. The positive pressure attained is sufficient to open the one-way valve for delivery of the milk into a milk collection container. In one embodiment, the positive pressure is in the range of 20 mm Hg to 40 mm Hg, typically about 25 mm Hg. In an alternative or additional embodiment, the one-way valve can be configured to crack open when the user breaks latch so that any milk remaining in the system will automatically drain into the milk container.

The present disclosure can establish a latch vacuum to cause the flange or skin contact member to seal to the breast. The latch vacuum established by the system is currently about 60 mmHg, but can be any value in a range of from about 20 mmHg to about 100 mmHg. Once the system has been latched to the breast via flange or skin contact member, the system then cycles between the latch vacuum and a target (also referred to as “peak” or “maximum”) suction level. Where the system is configured to maintain suction applied to the breast, with the minimum end of the suction cycle being the latch suction level (e.g., about 60 mm Hg), the nipple does not contract as much as it would with use of a prior art breast pump system. It has been observed that the nipple draws into the skin attachment member with the initial latch achievement in an analogous fashion as the formation of a teat during breastfeeding. Once the vacuum cycles between the latch and target vacuum levels, there is significantly less motion of the nipple back and forth with the vacuum changes. The nipple motion (distance between fully extended and fully retracted) during use of the system is typically less than about 2 mm, and in some cases less than about 1 mm. Accordingly, the system provides latching that is not only more like natural nursing, but the reduced nipple motion is also more like natural nursing as evidenced by scientific literature.

This greatly reduced motion of the nipple during cycling results from establishment of the latch at latch vacuum level, and then limiting the range of vacuum swing between latch vacuum (suction) and peak vacuum (suction). Typically the difference in vacuum between latch vacuum and peak vacuum is less than 200 mmHg, more typically less than 150 mmHg. In one example, the latch vacuum was 50 mmHg and the peak vacuum was 200 mmHg, resulting in a vacuum difference of 150 mmHg.

Limiting the nipple motion as described with use of the present system offers several benefits to the user. One benefit is that there is less friction on the side of the nipple against the flange wall, thereby greatly reducing the risk of irritation, skin damage, pain, swelling, etc. As a result, the present system is significantly more comfortable to use by a nursing mother, and this benefit is increasingly noticeable over repeated uses. By maintaining at least a latch suction level, breast pump system provides a more secure and persistent seal to the breast and significantly reduces the potential for leaks of air and/or milk. Because the nipple moves significantly less, this provides a more “natural” feel to the user that more closely simulates the feel of a nursing baby. Because the nipple travels less, this allows for the skin attachment member/flange to be designed as a lower profile component, as its length can be shorter since it does not need to accommodate the greater length in nipple movement experienced by prior art systems. This allows the overall amount of protrusion of the system from the breast to less than that in the prior art, as the overall length of the system is reduced by the reduction in length of the skin contact member/flange. Thus, the distance from the tip of nipple to exposed end of the housing the system is reduced.

During let down operation, the system operates to effect let down of the milk in the breast, prior to extraction, with a maximum suction target of up to 120 mmHg (typically, about 100 mmHg (−100 mmHg pressure)) to establish let down. The goal of letdown (or non-nutritive suction) is to stimulate the breast to express milk. The relatively shallow (small vacuum change range) and relatively fast frequency of the pumping during this phase are meant to mimic the initial suckling action of a child at the breast. This is because during let down phase, the suction pressure is not allowed to exceed the maximum let down suction of 110 mmHg or 120 mmHg, or whatever the maximum let down suction is set at. Therefore, the system is designed to reach-100 mmHg (a suction pressure of 100 mmHg) (or −120 mmHg, or whatever the maximum let down suction is designed to be).

During let down (non-nutritive) the system software and/or firmware communicates instructions to system motors based upon readings taken and communicated from the pressure sensing assembly so that the system is configured to operate between −60 mmHg and −100 mmHg in one example. The maximum latch suction pressure of −100 mmHg will be reached.

A sensor can be used to provide feedback to the controller for controlling the pumping cycles to achieve and/or maintain desired vacuum levels. Sensor is preferred to be a sensor capable of providing information usable to monitor the safety or function of the pump mechanism of system. As shown, sensor can be a non-contact sensor, meaning that it is not in fluid communication with the milk or vacuum space of the system. In an alternative approach, the sensor can be one that is configured to contact milk and be cleanable as needed. Here, a seal would be formed around the sensor element that is for example, positioned as a protrusion from the flange and to directly make measurements.

The positioning of the motor can be tracked and the vacuum force information provided by the sensor can be assessed to determine internal vacuum. By employing machine learning or supervised learning regression techniques, the system can be trained to interpret the motor positioning and sensor data (as well as motor speed or pump settings), while compensating for noise and hysteresis, to arrive at a pressure/vacuum level. More specifically, a neural net system or any mathematical regression of the data can be incorporated into system firmware so that sensor input can be translated to pressure/vacuum levels. In this regard, the system can include or communicate with a non-transitory computer readable medium having stored thereon instructions executable by a computing device of the system or external to the system to cause the computing devices to perform functions associated with and directed by the firmware.

Moreover, in one or more of the disclosed embodiments, the system controller can be configured to distinguish whether it has been attached to the left breast or the right breast of the user. This can be useful for tracking milk volume output per breast, per session, total daily volume per breast, etc. When using two of the pump systems, the tracking of data for each breast can still be maintained accurately, even when one of the pump systems is attached to the left breast during a current pumping session after having been attached to the right breast during a previous pumping session. In one embodiment, the pumping systems can establish current location (i.e., left or right breast) by receiving a signal from the other pumping system having been attached to the other breast. This established relative left-right locations of the two pumping systems, so that each system can accurately record as to whether milk is being extracted from the right breast or left breast. This identification is automatic, without any user input required and it also relieves the burden on the user to otherwise keep track of which pump system is placed on each breast and to maintain this order with each successive pumping session. Left and right pump labeling is also contemplated such as by placing markings on the system housing or cover jack, for example, near the power connector.

It is contemplated that the system is configured to pump into a sealed collection or container assembly, or one that incudes an integral valve or an otherwise airtight collection or container assembly, or combinations thereof. In this specific regard, the system can alternatively or additionally be closed and never vented to the atmosphere, and/or the system suction is only reduced through the flow of milk into the system. Thus, in at least one approach, milk or fluid that is pumped through the system is never exposed to new outside air from the environment once it enters the collection or container assembly. Accordingly, the orientation of the pump system or person has virtually no impact on the functioning of the system (i.e., no spills). The collection or container assembly can include a rigid or flexible sealing component, such as a ring or gasket into which the pump or container valve is pushed or twisted and sealed. The collection or container assembly can also include an opening or hole or structure that is pierced such that the container assembly seals about the member that goes into it.

In at least one embodiment, the pressure at which the valve to the container assembly opens to allow flow into the milk collection container is about 25 mm Hg. The valve can be configured and designed such that it allows fluid to flow through it when the pressure is positive, e.g., about 25 mm Hg, or some other predesigned “crack pressure”.

Once the flange or skin contact member is placed onto the main body/pump housing then pump power can be engaged. As the pump system goes through a power up routine, the controller reads the pressure sensor. The controller then calibrates the system such that the preload force or position or measured load equates to atmospheric pressure. Based upon a neural network or computer learning, such pressure readings in the system are observed during operation of the breast pump system and upon attachment to the breast.

The system can calculate the volume of milk pumped into system or alternatively the volume collected in the milk collection container assembly. As the pumping process is carried out, pumping/purging of milk into the milk collection container is tracked to calculate the change in volume of milk that is pushed into the milk collection container.

Various approaches to assessing milk volume can be included in the pump system. Certain approaches are described in co-pending U.S. application Ser. No. 15/448,716, the entirety of contents of which are incorporated herein by reference. A further approach to assessing expressed milk volume involves placing one or more disposable data collection devices on the mom or child. One specific approach involves creating a boundary on the skin of a breast and employing a fiducial to conveniently measure the change in size of the boundary. This change in size is then correlated to milk production to arrive at a volume of milk expressed or pumped. A crib or bassinet can also include sensors and communication hardware that communicate with the pump system so both assess and management milk consumption and needs, and baby health.

The system can calculate the pressure during operation in any of the manners described above. The suction (pressure) level can be varied as desired, and by continuously or repeatedly measuring/calculating pressure, the feedback provided by sensor(s) to controller provides a control loop that can be used to adjust the pump assembly and/or speed to vary the suction pressure to a level desired, or maintain a desired suction pressure in real time. Thus, controller can control the system to achieve any vacuum pressure pumping profile desired, and provide automatic, real time adjustments to maintain a desired vacuum pressure within the system. Also contemplated is responding in real time to maintain flow. This can accomplished independent or in conjunction with monitoring and regulating pressure in real time.

The controller tracks the motor driver position and calculates (or looks up) pressure based upon data received from sensor. The system controller or firmware is programmed with or retains information relating values detected by system sensors with driver positions and speed and system pressure. Thus, changes in motor position can be controlled by resulting changes in pressure calculated or looked up, relative to the pressure sought to be achieved. By using machine learning or supervised learning regression techniques, the system can be trained to interpret the motor positioning and pressure sensing (as well as motor speed or pump settings), while compensating for noise and hysteresis, to arrive at a pressure/vacuum level. More specifically, a neural net system or other mathematical regression can be incorporated into system firmware so that sensor input can be translated to pressure/vacuum levels.

In at least one contemplated embodiment, the system can be configured to communicate with a server, a remote computer, smartphone or other device such as through signal, such as by Wi-Fi, BLUETOOTH, BLUETOOTH Low Energy (BTLE), RFID, NFC or the like. In particular, one or more chips can be incorporated into the controller of the pumping system (by hard wire and/or wirelessly, preferably wirelessly) and configured to be in communication with an external computer. The controller and/or external computer communicates with the sensor(s)/chip(s) which indicate(s) when the system is in use, and can track usage. By tracking the times of use and/or number of uses, or even pump cycle counts, for example, the controller, or external computer can alert the user when it is time to change components or to report on usage aspects. In one particular aspect, the system can be equipped with an accelerometer to track and indicate the motion of the user and the angle at which the pump is used by the user. In this way, information such as the tracking of extraction date and time, volume extracted, etc. can be recorded and stored with regard to each milk collection container used with the system to extract milk, and a correlation can be made regarding the user's mobility or pumping angle respecting milk output and comfort. Moreover, the accelerometer can be employed to change pumping behavior based on the user's position for example, slowing or pausing the pump when the user is in a position or acting in a manner that may cause the system to leak or function improperly or less efficiently. Thus, the system can register individual milk collection containers, so that the user can readily identify when milk in each container was collected, the volume in each container, etc. The breast pump system can record the volume of milk in any given container during a pumping session. The data recorded can be sent to an external computer and/or over the Internet, either automatically or manually. Thus, user data and trends can be collected, stored and analyzed as they relate to volume (from each breast and in total), as can be the number of sessions on several dimensions (per day, week or month). Data and analytics can thus be provided to a user concerning pumping sessions.

In one particular approach, at least the session start time, the session end time and total volume of milk extracted from the breast can be stored and tracked. Sessions can be defined as the commencement of latch and can continue up to and through pauses of up to 5 minutes, for example. Thus, a pause of over 5 minutes can be defined as the end of the previous session. A language protocol is generated so that there is a two-way communication between an external device or program and the breast pump. That is, both the pump and external device can create and understand and are responsive to specific messages. Further, live data and historical data can be treated differently, and their data streams maintained separately. Live updates are generated and stored at the pump and are available by the external device to retrieve (for example, up or down button activations or volume updates). Accordingly, such live data can be reflected on and update the screen of the external device. Historical data is stored inside the pump in a stream and the pump can communicate with this stream to extract or act upon the same. An internal pump memory such as a disc within a chip or other internal flash stores, communicates with the pump so that session data is written to an internal history log. At the end of a session, for example, the pump will write the session data to its internal history log and the external device will ask if there is any data and if the pump indicates that there is, then the external device will download this historical data to update its non-live view screens. The external device can also make this same query after an extended time and then download multiple session data, and the query also can be made during a session. In one particular embodiment, as much as 600 sessions of data can be stored.

In one or more embodiments, the system can additionally or further include structure configured to accomplish or functionality operating as an Active Pause Mode, that allows the system to maintain latch vacuum, while remaining (especially under no/low flow) virtually silent. Such a system stays much quieter than pump mode, but ensures the system does not fall off the breast. It can be employed by the user mom when she needs to interact with others and does not want them to hear the pump, or for some other reason where she might not be ready to remove the device but does not want active pumping either.

A remote user interface on an external device can take a myriad of forms. A user profile can be created for a child and linked to a child's birthdate. Other details such as tracking the child's age when use of the system commenced can be gathered so that analytics pertinent to the child's age can be generated. In this way, pump performance can be tracked to the growth of the child. Reminders can be entered into the system so that the user can focus on matters other than breast pumping. Notifications can be keyed to time or volume of milk pumped while both of such criteria as well as battery life can be tracked and reflected on the remote computer. Easily understood and convenient graphics are contemplated for expressing status such as curved hemispherical strips reflecting volumes pumped for each pump system, the same information also being shown in numerical form. Timing countdowns as well as information from one or more previous sessions can also be graphically displayed for effectively communicating with a user. The ability to remotely begin a new session can also be made available to the user.

The remote user interface, whether provided as an App, on a cell phone, computer or other computing device, can also include specific user control functionality, and various related easy to understand displays. In one or more approaches, the amount of milk pumped is tracked by day, and an option is provided to the user to set a session tracker by day. The amounts pumped are also tracked by breast. A user can set one or more of time and volume of pumping by breast for one or more pumping sessions. A volume target can be set by the user by various increments such as 0.1 ounces. This setting can be set and saved, or canceled. The user can then control whether to pump with one or both breasts, and then the system starts tracking pumped volume. As pumping progresses, easily readable curved bars reflect the amount of volume being pumped by each breast, the bars becoming thicker as more volume is pumped. The user can adjust suction levels for one or both of the pumps attached to a breast to coordinate pumping or to otherwise pump as desired. After reflecting the changes in suction level, the user can return the system to tracking volumes pumped by breast, and an indication of volume remaining to be pumped is also provided. Once the pumping target is met, such as a target volume, the user interface will indicate that the session has been completed. Thereafter, an updated set tracker is presented, with an ability to set further pumping schedules. The user can then select an option to depict a summary of pumping or a history of pumping. The data provided by the user interface can include bar graphs and numerical data showing pumping by day and by breast and session times and number of session. Additionally, circles can be sized to represent relative amounts of pumping by date, and color coated by breast.

In still yet further embodiments and approaches, the pump system can alternatively or additionally include built-in or computer or App based functionality to de-stress the user's life, empower the user to better take care of the nursing baby's health, maximize the user's mobility and freedom, and support all that is involved in becoming or being a parent. In these regards, pump system structure and functionality can include one or more of keying on pain points, physical conditions, sleep, pain relief, and post partum issues, tracking sleep, sensing and tracking baby vitals and movement, focusing on connected health with the mom as the caregiver, and/or providing education, guidance or instruction on movement and ways to carry a baby, fertility, post baby needs, health of the mom, ultrasound and fertility. The pump system can additionally include App integration with smart bottles, smart scales or the like to facilitate managing overall baby health and nutrition. App updates can additionally be provided about stimulation and letdown, and timing of pumping based upon such information, such as suggestion to begin pumping. System structure and functionality can also involve updating pumping profiles based upon baby age and needs, developing pumping functionality which enhance milk production, enhance efficiency or comfort or better mimic baby. Data can be stored in the cloud for analysis, and additional functionality can be provided to modify speeds and alternated between and among customized modes and profiles. Additional or a myriad of sizes of flanges and bag or container assemblies can be provided to the user as can nighttime pump functionality or programming including automated sessions with starts and stops.

Inventory management is further functionality that is provided as part of the structure of the pump system. In connection with the same, container assemblies can include structure that is scannable or which otherwise communicates with the inventory management system (e.g., via bar codes, RFID chips). Further, operative communication structure can be provided so that the user can transmit data with and between a baby-center platform that stores data thus facilitating an avenue for the effective management of the baby's nutrition, and links can be made to automatically communicate with milk banks and donation centers. Additionally, a caregiver data share system can be included within the functionality and structure of the pump system. Texting is added to other forms and avenues for communicating such important and useful information.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the present disclosure.

That which is claimed is:

1. A wearable system to pump fluid from a breast, the system comprising:
a flange configured and dimensioned to form a seal with the breast, the flange including a nipple tunnel;
a lid sized and shaped to receive the flange;
a diaphragm;
a first chamber, the first chamber defined by the lid and diaphragm;
a pump assembly configured to flex the diaphragm and create a vacuum within the first chamber; and
a milk collection container assembly configured to receive pumped milk;
wherein the diaphragm seals at multiple locations with one or more of the lid, the pump assembly and the flange.

2. The system of claim 1, wherein the volume equalization and diffusing chamber functions to one or more of dampen sound or accomplishes equalizing a distribution of vacuum within a nipple tunnel.

3. The system of claim 1, further comprising a controller that controls vacuum levels within the system.

4. The system of claim 1, wherein an external surface of the nipple tunnel includes an annular ring.

5. The system of claim 1, wherein the annual ring includes one or more cut-outs form therein.

6. The system of claim 1, wherein the diaphragm surrounds the nipple tunnel.

7. The system of claim 1, further comprising a volume equalization and diffusing chamber, the volume equalization and diffusing chamber defined by the lid and flange, wherein the diaphragm functions to create a vacuum within the first chamber and the volume equalization and diffusing chamber conditions the vacuum being applied within the nipple tunnel.

8. The system of claim 1, wherein the diaphragm defines a frusto-conical profile.

9. The system of claim 1, wherein the milk collection container includes one or more splash ribs configured to inhibit flow of collected milk upwardly along sides of the milk collection container.
